SEQUENCE LISTING (2 sequences)

SEQ ID NO: 1 - protein, 366 aa, Homo sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 2 - protein, 95 aa, Artificial sequence
  Description of artificial sequence  synthetic peptide AA4RP
ATQARKGFWDYFSQTSGDKGRVEQIHQQKMAREPATLKDSLEQDLNNMNKFLEKLRPLSGSEAPRLPQDPVGMRRQLQEELEEVKARLQPYMAEA

(19) United States
(12) Patent Application Publication
(10) Pub. No.: US 2004/0197823 A1
(43) Pub. Date: Oct. 7, 2004

(54) Compositions and methods for aa4rp assay

(76) Inventors: Jamila Najib, Santes (FR); Zouher Majd, Marc en Baroeul (FR)

Correspondence Address: NIXON & VANDERHYE, PC, 1100 N GLEBE ROAD, 8TH FLOOR, ARLINGTON, VA, 22201-4714, US

(21) Appl. No.: 10/487,096

(22) PCT Filed: Sep. 6, 2002
(86) PCT No.: PCT/FR02/03041

(30) Foreign Application Priority Data
Sep. 7, 2001 (FR) 0111598
Aug. 12, 2002 (FR) 0210205

(51) Int. Cl. G01N 33/53; C12P 21/04; C12N 9/22; C07K 16/40

(52) U.S. Cl. 435/7.1; 435/70.21; 530/388.26; 435/199

(57) ABSTRACT

The invention relates to compositions and methods for assaying or detecting “Apolipoprotein AIV-related protein” (AA4RP) in samples. In particular, it relates to a method allowing the direct measurement of AA4RP in biological samples. The invention also relates to synthetic products of AA4RP, the corresponding antibodies and the kits containing them, and their uses for the detection, quantification of AA4RP in a sample, or yet the quantification of atherogenic and non-atherogenic lipoparticles in a sample.

The invention relates to compositions and methods for assaying or detecting “Apolipoprotein AIV-related protein” (AA4RP). In particular, it relates to a method allowing the direct detection and quantification of AA4RP. The invention also relates to synthetic products of AA4RP, the corresponding antibodies, the kits containing them, and their uses to detect and quantify AA4RP in a biological sample.

The elevation of plasma triglyceride levels is one of the determining factors in the development of cardiovascular diseases which today are the leading cause of death worldwide (Hokanson and Austin 1996). Studies have shown that alterations in the concentration or structure of apolipoproteins are linked to abnormal lipid levels and an increased risk of atherosclerosis (Rubin, Krauss et al. 1991; Plump, Smith et al. 1992; Duverger, Tremp et al. 1996; Huang, Liu et al. 1998; Lusis 2000; Rubin and Tall 2000). One very pertinent example of the involvement of apolipoproteins in the plasma triglyceride balance is that of AA4RP. This recently discovered apolipoprotein has been shown to be an important factor in the regulation of triglyceride metabolism in mice and humans (Pennacchio, Olivier et al. 2001). Mice transgenic for human AA4RP showed a three-fold reduction in plasma triglycerides as compared to control mice. Conversely, knock-out mice which do not possess the gene coding for AA4RP had plasma triglyceride levels four-fold higher than control mice. The variations in triglyceride levels observed in transgenic mice and in AA4RP knock-out mice are in line with the variations in the levels of apolipoprotein CIII (apo CIII), known to be a positive indicator of triglyceride levels due to the fact that this apolipoprotein inhibits the hydrolysis of triglycerides by lipoprotein lipase (LpL). The apo CIII concentration in plasma was reduced in mice transgenic for human AA4RP fed a lipid-rich diet whereas apo CIII levels in knock-out mice were increased, indicating that one of the mechanisms of action of AA4RP might be to regulate expression of the apo CIII gene and thus confirming the positive role of AA4RP in the elimination of triglycerides.

In man, a very close correlation has been demonstrated between AA4RP polymorphism and triglyceride levels. In fact, the minor alleles SNPs1-3 of the AA4RP polymorphism are associated with high triglyceride levels. A representative result is the 30% increase in triglyceride level in individuals with one of these alleles compared to subjects who are homozygous for the wild-type alleles (Pennacchio, Olivier et al. 2001).

The results obtained in mice and in humans clearly reveal the importance of the role of AA4RP in triglyceride homeostasis and suggest that AA4RP may be used as a marker for the prognosis or diagnosis of hypertriglyceridemiae. Development of a method by which to detect and/or quantify AA4RP is therefore of particular relevance.

For various reasons, it is quite difficult to measure the concentrations of AA4RP in plasma. In the first place, AA4RP shares 27% identity and 48% similarity with apolipoprotein AIV (apo AIV). In the second place, AA4RP is present at very low concentration in plasma. It was therefore essential to develop a specific and sensitive method for quantifying and/or detecting this apolipoprotein.

Said protein comprising 366 amino acids has the following primary structure (SEQ ID NO: 1).

MASMAAVLTWALALLSAFSATQARKGFWDYFSQTSGDKGRVEQIHQQ
KMAREPATLKDSLEQDLNNMNKFLEKLRPLSGSEAPRLPQDPVGMRR
QLQEELEEVKARLQPYMAEAHELVGWNLEGLRQQLKPYTMDLMEQVA
LRVQELQEQLRVVGEDTKAQLLGGVDEAWALLQGLQSRVVHHTGRFK
ELFHPYAESLVSGIGRHVQELHRSVAPHAPASPARLSRCVQVLSRKL
TLKAKALHARIQQNLDQLREELSRAFAGTGTEEGAGPDPQMLSEEVR
QRLQAFRQDTYLQIAAFTRAIDQETEEVQQQLAPPPPGHSAFAPEFQ
QTDSGKVLSKLQARLDDLWEDITHSLHDQGHSHLGDP.

Said protein corresponds to the sequence of the RAP3 protein previously described as potentially involved in liver regeneration (Genbank access number: AF202889.1: Van der Vliet H. N., Groenink M., Leegwater A. C. J. and Chamuleau R. A. F. M. Submitted (09-NOV-1999) Experimental Hepatology, Academic Medical Center, Meibergdreef 9, Amsterdam 1105 AZ, Netherlands). It also corresponds to the AA4RP protein (Apolipoprotein AIV-Related Protein) in international patent application WO01/00803 A2 (SEQ ID NO: 3 of said application).

The invention provides a strategy for producing a synthetic peptide specific of AA4RP (which is used for production of specific anti-AA4RP antibodies) and a novel immunoenzymatic method by which to detect and directly quantify this apolipoprotein. The quantification of AA4RP is advantageously carried out by the ELISA method (Enzyme Linked Immuno-Sorbant Assay) of the sandwich type. The anti-AA4RP antibody is coated on a solid support for capture of the protein, then the same antibody, now labelled with an enzyme, is used as antibody to detect AA4RP bound to the first antibody. The detection phase is carried out in the presence of a substrate of the enzyme and the intensity of the reaction that develops between the enzyme and its substrate is directly proportional to the AA4RP concentration. To detect, quantify or purify AA4RP using specific anti-AA4RP antibodies, either polyclonal antibodies, or monoclonal antibodies or a mixture thereof may be used.

The invention also describes said antibodies, the kits containing them and their uses for the detection, quantification or purification of AA4RP in samples, particularly serum or plasma. Said antibodies also offer a novel approach for modulating AA4RP activity in vitro or in vivo, and for regulating lipid metabolism in a subject.

A first specific object of the invention relates to a substantially pure synthetic peptide specific of AA4RP, comprising the sequence SEQ ID NO: 2 or an immunogenic fragment or a derivative of said peptide.

Another object of the invention relates to a method for producing anti-AA4RP antibodies comprising an immunization step with a synthetic peptide specific of AA4RP such as defined hereinabove. This invention also comprises antibodies prepared according to said method, and, more generally, antibodies able to bind a peptide such as defined hereinabove as well as fragments or derivatives of such antibodies.

Another aspect of the invention concerns a method for detecting or assaying AA4RP in biological samples (particularly in prepared lipoparticles or in whole plasma or serum samples), using an antibody (including a fragment or a derivative thereof) such as defined hereinabove.

Another object of the invention relates to a method for detecting the presence of a predisposition or a risk of developing a disorder of lipid metabolism in a subject, comprising detecting AA4RP in vitro, in a sample from said subject, by means of an antibody (including a fragment or a derivative thereof) such as defined hereinabove.

A further object of the invention relates to a method for detecting or monitoring in a subject the cellular uptake of triglyceride-rich lipoproteins and HDL, comprising detecting in vitro particles containing AA4RP by means of an antibody (including a fragment or a derivative thereof) such as defined hereinabove.

Another object of the invention concerns a method for detecting and/or monitoring the formation, development or progression of atherosclerosis in a subject, comprising detecting in vitro, in a sample from said subject, the quantity or level of AA4RP by means of an antibody (including a fragment or a derivative thereof) such as defined hereinabove.

The subject is generally a mammal, preferably a human being, even more preferably a subject at risk of developing cardiovascular diseases linked to a dyslipidemia (more preferably a hypertriglyceridemia) such as a coronary disease for example.

Another object of the invention relates to a pharmaceutical composition comprising an antibody (including a fragment or a derivative thereof) such as defined hereinabove and a pharmaceutically acceptable excipient or vehicle.

Specific Peptides

As indicated hereinabove, one aspect of the invention concerns a substantially pure synthetic peptide specific of AA4RP comprising the sequence SEQ ID NO: 2 or an immunogenic fragment or a derivative of said peptide. The term “substantially pure” indicates that the peptide is essentially devoid of amino acid sequences present in apo AIV and/or of contaminating proteins normally present or associated with lipoparticles, such as apo A1 in particular. The term “synthetic” indicates that the peptide is not a molecule obtained naturally but rather it has been prepared by an artificial method (e.g., chemical synthesis, assembly, etc.) particularly as described in the examples. In this respect, the synthetic peptide specific of AA4RP of the invention is preferably essentially not glycosylated. An inventive peptide generally contains fewer than 200 amino acids, more preferably fewer than 150 amino acids, even more preferably fewer than 120 amino acids.

The invention now demonstrates that synthetic peptides specific of AA4RP may be produced and used to generate specific anti-AA4RP antibodies. The invention further demonstrates that said antibodies can bind specifically to AA4RP obtained either naturally or in the form of a soluble antigen, or included in lipoparticles. The invention also demonstrates that said antibodies can bind to different lipoparticles containing AA4RP (VLDL and HDL). Said synthetic peptides specific of AA4RP and their corresponding antibodies thus represent novel products which are particularly advantageous for the detection of AA4RP and the specific quantification of said apolipoprotein.

More particularly, a preferred synthetic peptide specific of AA4RP according to the invention comprises the sequence SEQ ID NO: 2, such as shown hereinbelow, or an immunogenic fragment or a derivative of said peptide.

SEQ ID NO:2

ATQARKGFWD YFSQTSGDKG RVEQIHQQKM AREPATLKDS LEQDLNNMNK FLEKLRPLSG SEAPRLPQDP VGMRRQLQEE LEEVKARLQP YMAEA

Said peptide fragment of 95 amino acids corresponds to residues 20 to 114 of the mature human AA4RP sequence.

As illustrated in the examples, said peptide (comprising the sequence SEQ ID NO: 2) may be prepared in a particularly advantageous manner by solid phase synthesis, more particularly by using a Boc/Bzl strategy (Merrifield 1963).

The term “derivative” encompasses peptides comprising one or more mutations, substitutions, deletions and/or additions of one or more amino acid residues and having substantially the same antigenic specificity. Typical examples of derivatives include sequence variations due to AA4RP polymorphism, splicing, and the like. Especially preferred derivatives comprise a sequence SEQ ID NO: 2 or a modified sequence comprising at most 5 amino acids different from those present in sequence SEQ ID NO: 2. The additional residues may be transport or binding sequence residues, protector groups, and the like. Furthermore, the peptide may be modified for example, by a chemical, physical and/or enzymatic route, so as to increase its stability, increase its immunogenicity or yet incorporate a “Tag” or any other carriage molecule or marker, etc. Examples of such modifications include glycosylation, addition of a carriage, a marker (e.g., radioactive or enzymatic label, etc.), and the like.

The term “fragment” denotes any peptide comprising from 5 to 95 consecutive residues of the sequence SEQ ID NO: 2, preferably from 10 to 95. The term “fragment” includes any portion of sequence SEQ ID NO: 2 comprising an epitope.

A specific object of the invention relates to a synthetic peptide specific of AA4RP wherein it comprises epitopes specific of AA4RP and wherein it is devoid of epitopes specific of apo AIV, and wherein it comprises the sequence SEQ ID NO: 2 or a fragment or a derivative of said sequences.

The peptides may be soluble, purified or complexed with a carrier molecule, such as KLH (Keyhole Limpet Hemocyanin) or serum albumin for example, or yet any other inert molecule (e.g. synthetic) such as a bead, etc. According to a particular embodiment of the invention, the peptides are coupled to a carrier molecule. In particular this is the case when they are used for production of antibodies. The coupling may be carried out according to conventional methods known to those skilled in the art (Vaitukaitis, Robbins et al. 1971) (Bassiri 1979).

The peptides may also be conjugated or coupled with a heterologous peptidic molecule, such as a biologically active molecule for example. The heterologous nature denotes any peptide which does not originate from an AA4RP molecule.

A specific object of the invention concerns a composition wherein it comprises a synthetic peptide comprising the sequence SEQ ID NO: 2, and wherein it is devoid of other proteins and in particular of apolipoproteins.

The peptides may be used in the framework of screening methods for titration tests, as controls, standards or for test calibration. They may also be used to modulate the activity of AA4RP. They are also particularly useful for producing anti-AA4RP antibodies.

Antibodies

In this respect, another object of the invention concerns any antibody able to bind to a peptide such as defined hereinabove. The antibody may be polyclonal or monoclonal. Moreover, the term antibody generally includes any antibody fragments or derivatives, in particular fragments or derivatives of said monoclonal or polyclonal antibodies displaying substantially the same antigenic specificity. The latter comprise antibody fragments (Fab, Fab′2, CDRs, etc.), humanized antibodies, polyfunctional antibodies, single chain antibodies (ScFv), etc. The antibodies of the invention may be produced by conventional methods, comprising immunizing an animal and recovering the serum (polyclonal) or spleen cells (for production of hybridomas by fusion with suitable cell lines).

Methods of production of polyclonal antibodies from various species including mice, rodents, primates, horses, pigs, rabbits, fowl, etc. may be found for example in Vaitukaitis et al. (Vaitukaitis, Robbins et al. 1971). The antigen is combined with an adjuvant (e.g., Freund's adjuvant) and administered to an animal, typically by subcutaneous injection. Repeated injections may be given. Blood samples are collected and immunoglobulins or serum are isolated.

Methods for producing monoclonal antibodies from different species may be found for example in Harlow et al. (Harlow 1988) or in Kohler et al. (Kohler and Milstein 1975). Said methods comprise immunizing an animal with an antigen, then recovering spleen cells which are then fused with immortalized cells such as myeloma cells. The resulting hybridomas produce monoclonal antibodies and may be selected by limit dilution to isolate individual clones. The antibodies may also be produced by selection of combinatorial immunoglobulin libraries, such as disclosed for example in Ward et al. (Ward, Gussow et al. 1989).

Preferred inventive antibodies are prepared by immunization with a substantially pure synthetic peptide specific of AA4RP such as described hereinabove, preferably comprising the sequence SEQ ID NO: 2 or an immunogenic fragment or a derivative of said peptide, e.g. a subfragment comprising at least one epitope.

Fab or Fab′2 fragments may be produced by digestion with a protease according to conventional methods. Humanized antibodies may be prepared by one of the methods described, for instance, in Riechmann et al. (Riechmann 1988) (Jones 1986).

The invention also concerns a method for producing anti-AA4RP antibodies, comprising injecting a peptide having the sequence SEQ ID NO: 2 or an immunogenic fargment or a derivative of said peptide in a non-human animal and recovering antibody or antibody-producing cells. The method advantageously allows the production of specific antibodies. Specificity may be verified by demonstrating the absence of a cross reaction with other circulating blood proteins. More generally, specificity indicates that the antibodies are able to bind to AA4RP with higher affinity than to any other antigen. As illustrated in the examples, the polyclonal antibodies of the invention may be used for detecting and/or assaying AA4RP with high efficiency.

The antibodies may be coupled to heterologous fragments such as toxins, markers, medicaments or any other therapeutic agent, covalently or not, either directly, or by means of coupling agents. The markers may be selected in the group consisting of radiolabels, enzymes, fluorescent agents, magnetic particles, etc. Preferred toxins are exemplified by diphtheria toxin, botulism toxin, and the like. Medicaments or therapeutic agents are selected in particular from among lymphokines, antibiotics, anti-sense sequences, growth factors, and the like. Methods by which to couple antibodies with said heterologous fragments are illustrated for example in patent U.S. Pat. No. 4,277,149 and in patent U.S. Pat. No. 3,996,345.

The inventive antibodies have many uses including therapeutic, prophylactic, diagnostic applications, for purification, detection, etc.

In vitro, they may be used as screening agents or for purifying antigen from various samples, including different biological samples (e.g., blood samples). They may also be used to detect or quantify the presence or the quantity of AA4RP in lipoparticles in a sample collected from a subject, typically, a blood sample from a mammal or, preferably, from a human being.

Detection/Assay

In this respect, another object of the invention concerns a method for detecting, measuring, assaying or quantifying AA4RP in a biological sample, comprising contacting the sample with an antibody such as defined hereinabove (including fragments or derivatives thereof) and detecting, measuring, assaying or quantifying (the presence) of antigen-antibody immune complexes. Typically, said method enables AA4RP levels to be determined in a sample, by comparison with standard conditions or with a calibration curve, for example.

The determination of immune complexes may be carried out by conventional techniques such as direct or competitive immunological methods, for example the RIA method (Radio Immuno Assay), EIA (Electro Immuno. Assay), by nephelometry, turbidimetry, quantitative immunoblot, calorimetry, interferometry, surface resonance, force field measurement, other biosensors under development, etc. However, an especially preferred object of the invention concerns a method based on the ELISA sandwich assay. As a matter of fact, as indicated hereinabove, the antibodies are specific and can recognize AA4RP in a sample, thus allowing a precise, sensitive and efficient assay of AA4RP with a polyclonal anti-AA4RP antibody (or a mixture of monoclonal antibodies).

A more preferred object of the invention therefore concerns a method for detecting, measuring, assaying or quantifying AA4RP in a biological sample, comprising contacting the sample (or dilutions thereof) with a first antibody immobilized on a support, called a capture antibody, said antibody being defined hereinabove (including fragments or derivatives thereof), and revealing any antigen-antibody immune complexes formed by means of a second labelled antibody, called detection antibody, said antibody being such as defined hereinabove (including fragments or derivatives thereof).

In a particularly preferred embodiment, the capture and detection antibodies are polyclonal antibodies. They may be the same polyclonal antibody. The capture antibody may be immobilized opn any type of support, particularly plastic, such as a plate, bead, column, gel and the like. In an advantageous manner the support is a multiwell plate. Immobilization is typically achieved by adsorption of the antibody on the surface of the wells. It is understood that any other method of coating may be used, direct or indirect, covalent or not. The capture antibody is typically used in soluble form. It is first labelled so as to enable its detection and quantification. There are various types of labelling, such as radioactive, fluorescent, enzymatic, luminescent labelling, etc. In a specific embodiment, the labelling is an enzymatic labelling, and the immune complexes are revealed by measuring enzymatic activity. A specific example of enzymatic labelling is peroxidase, the activity of which may be visualized in the presence of the OPD substrate for example.

A particularly preferred object of the invention concerns a method for detecting, measuring, assaying or quantifying AA4RP in a biological sample, comprising contacting the sample (or dilutions thereof) with a support on which a polyclonal capture antibody such as defined hereinabove is immobilized, under conditions which allow formation of specific immune complexes, and revealing the antigen-antibody immune complexes eventually formed by means of a labelled polyclonal detection antibody, said polyclonal antibody defined as hereinabove.

The method may be carried out on different biological samples, including plasma, serum, interstitial fluid, cell culture supernatant, etc. The sample may be collected from a subject (e.g. a human subject) and used directly in the test. Alternatively, the sample may be diluted and/or stored (for example frozen) for testing at a later date. The invention also provides for the measurement of AA4RP concentrations in lipoparticles through the use of specific anti-AA4RP antibodies, with very high sensitivity, reproducibility and repeatability.

Detection may be carried out under different experimental, clinical, epidemiological, prognostic and diagnostic conditions. In particular, the method may be used to detect the predisposition of certain subjects to develop disorders of lipid metabolism.

A specific object of the invention thus relates to a method for detecting the presence of a predisposition or a risk of developing a disorder of lipid metabolism in a subject, comprising detecting in vitro or measuring in vitro, on a sample taken from the subject, lipoparticles containing AA4RP, using an antibody such as defined hereinabove (including fragments or derivatives thereof). The AA4RP levels (by comparison with a mean value in normal subjects) may be characteristic of a high risk of developing disorders of lipid metabolism.

Another object of the invention concerns a method for detecting or monitoring cellular uptake of HDL and triglyceride-rich lipoproteins in a subject, comprising detecting in vitro the quantities of AA4RP present in lipoparticles, using an antibody such as defined hereinabove (including fragments or derivatives thereof).

Another object of the invention relates to a method for monitoring a treatment for correcting disorders of lipid metabolism in a subject, comprising detecting AA4RP levels in vitro, in a sample from said subject, using an antibody such as defined hereinabove (including fragments or derivatives thereof), after administering said treatment to said subject. The efficacy of the treatment is correlated with the AA4RP level in the subject. These results can be correlated with the ability of the treatment to regulate the levels or activity of AA4RP or the ability to restore a normal AA4RP level or activity in a subject.

A further object of the invention concerns a method for evaluating the physiological state of a subject, e.g. the level of lipid metabolism in a subject, comprising detecting AA4RP levels in vitro or ex vivo in a sample from said subject, using an antibody such as defined hereinabove (including fragments or derivatives thereof).

As indicated hereinabove, said methods may be carried out on differnt samples (typically on plasma or serum) and by RIA, EIA, nephelometry, turbidimetry, quantitative immunoblot, calorimetry, interferometry, surface resonance, force field measurement, other biosensors under developmment, etc. or preferably by means of an ELISA sandwich assay.

The invention also comprises a pharmaceutical composition comprising an antibody such as defined hereinabove and possibly a pharmaceutically acceptable excipient or vehicle.

The invention further concerns a kit comprising a peptide or an antibody such as described hereinabove. The kit may be used for detection or quantification of AA4RP in any sample.

Other aspects and advantages of the invention will be described in the following examples, which are given for purposes of illustration and not by way of limitation.

### LEGENDS OF FIGURES

FIG. 1:

Antibody specificity.

SDS PAGE control of anti-AA4RP antibody specificity and demonstration of AA4RP distribution in VLDL and HDL.

FIG. 2:

ELISA test on AA4RP

The calibration curve was plotted as OD versus concentration of peptide 20-114 (♦), on plasma from normolipidic subjects (▴) and HDL prepared from plasma from normolipidic subjects (●) according to the protocol described in example 3.

FIG. 3:

AA4RP concentrations in plasma calculated from a calibration series of peptide 20-114 or pooled plasma from normolipidic subjects by ELISA FIG. 4:

ELISA assay of AA4RP in non-transgenic C57 BL/6 mice and FVB mice, AA4RP knock-out (KO) mice with a FVB genetic background and mice transgenic for human AA4RP (FVB genetic background).

### EXAMPLES

### Example 1

### Peptide Sequence

1. Choice of Suitable Peptide Sequence

The following peptide fragment was synthesized. Said fragment was defined by using different algorithms to predict flexibility, hydrophilicity, antigenicity, and secondary structures. Said fragment containing 95 amino acids corresponds to residues 20 to 114 of the sequence SEQ ID NO: 1.

ATQARKGFWD YFSQTSGDKG RVEQIHQQK AREPATLKDS LEQDLNNMNK FLEKLRPLSG SEAPRLPQDP VGMRRQLQEE LEEVKARLQP YMAEA

2. Peptide Synthesis.

The peptide was synthesized by solid phase synthesis (Merrifield 1963) on an ABI 431 A automatic synthesizer (Applied Biosystems Inc., California, USA) using a Boc/Bzl strategy on 0.5 mmol (0.57 mmol/g) MBHA resin. Each amino acid was coupled twice in the presence of dicyclohexylcarbodiimide/hydroxybenzotriazole without capping. Side chain protector groups were as follows: Arg(Ts), Asp(Ochex), Glu(Ochex), Lys(2-Cl-Z), His(Dnp), Ser(Bzl), Thr(Bzl), Met(O), Trp(formy) and Tyr(Br-Z).

The Dnp group on the histidine residue was eliminated from the peptide before cleavage from the support by treatment in the presence of 10% β-mercaptoethanol, 5% diisopropylethylamine in DCM medium for 2 hours then in NMP medium for 2 hours. The peptidyl resin was treated with 50% TFA in DCM medium for 20 minutes to eliminate the terminal amino acid Boc. The peptide was cleaved from the resin and simultaneously deprotected according to a slow and rapid HF procedure: the resin (1 g) was treated with anhydrous HF (2.5 ml) in the presence of p-cresol (0.75 g), p-thiocresol (0.25 g) and dimethylsulfide (0.5 ml) at 0° C.

Three hours later, the hydrogen fluoride and dimethylsulfide were eliminated by vacuum evaporation and the residual scavengers and secondary products were extracted with diethyl ether. The reaction vessels were reloaded with p-cresol (0.75 g), p-thiocresol (0.25 g) and 10 ml of anhydrous HF and the mixture was incubated at 0° C. for 1.5 hours. Hydrogen fluoride was eliminated by evaporation and the residue was mixed in the presence of diethyl ether. The residue was filtered, washed with diethyl ether and extracted with 200 ml of a 10% aqueous solution of acetic acid, then freeze-dried.

3. Mass Spectrometry

The molecular mass was determined on an ion electrospray mass spectrometer. The electrospray spectrum was obtained by using an API apparatus (Perkin-Elmer-Sciex) on a single quadrupole ion electrospray mass spectrometer, equipped with an ion spray (electrospray assisted by a nebulizer).

4. Immunization

The peptide was emulsified in complete Freund's adjuvant and injected subcutaneously in rabbits at a dose of 0.5 mg per injection for the first two injections, followed by a booster dose of 0.25 mg of peptide in the same adjuvant every two weeks.

### Example 2

### Isolation and Characterization of Rabbit Specific Anti-AA4RP Antibodies

1. Isolation of Anti-AA4RP Antibodies

Polyclonal antibodies were isolated by precipitation with 27% sodium sulfate then purified by affinity chromatography on activated Sepharose 4B gel (Pharmacia, Uppsala, Sweden), coupled with the AA4RP peptide residue 20 to 114 AA (Axen, Porath et al. 1967). Proteins not retained on the antigenic gel were eliminated by washing with phosphate buffered saline (PBS: Phospate 50 mmol/L, pH 7.2, NaCl 150 mmol/L). Fractions not specifically bound on the AA4RP gel were eliminated with PBS 25 mmol/L. AA4RP-specific polyclonal IgG were eluted with 0.2 M glycine pH 2.8. The purified antibodies were immediately dialyzed against PBS 10 mmol/L then concentrated by ultrafiltration on an Amicon system (cutoff 100 kD) (Amicon, Dr. Bervely, MA, USA), assayed for protein content (Lowry O. H. 1951), then stored in 1 ml aliquots (1 mg) at −30° C.

2. Western Blot Analysis

2.1—Protocol:

Antibody purity and specificity were analyzed by western blot (Towbin, Staehelin et al. 1979). Human HDL, LDL and VLDL particles were subjected to denaturing SDS-PAGE electrophoresis (5 to 24%), then transferred to a nitrocellulose membrane and reacted with purified anti-human-AA4RP antibody. Immunoreactive proteins were visualized with a horseradish peroxidase-conjugated anti-IgG polyclonal antibody (Sanofi-Diagnostics Pasteur, Marnes-la-Coquette, France). The reaction was developed by chemiluminescence (Amersham, Pharmacia, Biotec).

2.2—Results

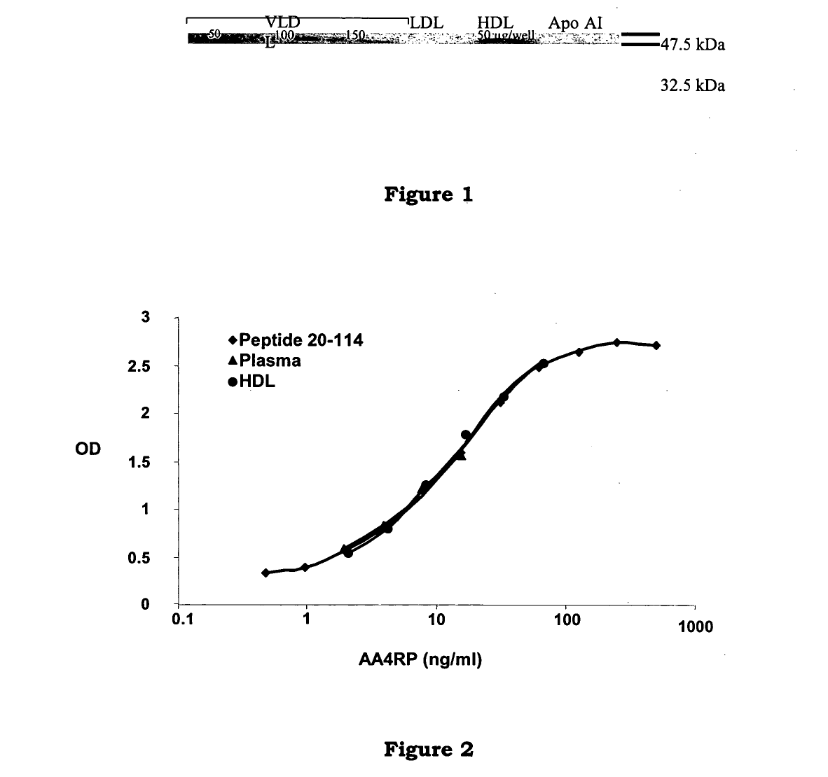

The results are presented in FIG. 1. It can be seen on this figure that the specific band revealed by the anti-AA4RP antibody is located between the 32.5 and 47.5 kDa molecular weight markers.

2.3—Interpretation

The immunoblot results on the different human lipoproteins show that AA4RP is localized in VLDL and HDL, primarily in HDL.

The presence of AA4RP in VLDL would explain the role of this apolipoprotein in metabolic regulation of these triglyceride-rich lipoproteins, and thus the modulation of the concentration of these atherogenic lipids.

The localization of AA4RP in HDL is undoubtedly related to the role of these particles in reverse cholesterol transport. In fact, the majority of apolipoproteins located in HDL promote uptake of cell-derived cholesterol by HDL and transfer to the liver where it is eliminated.

### Example 3

### ELISA Assay of AA4RP (Sandwich Type)

1. Reagents and Materials Used for the ELISA Test

1.1—Capture Antibody

The anti-AA4RP mother solution was concentrated to 1 mg/ml, the coating (fixation of antibody in the wells of a 96-well plate) was carried out at 10 μg/ml, the antibody was diluted in phosphate buffered saline (0.1 M PBS, 0.15 M NaCl, pH 7.2 to 7.4).

1.2—Detection Antibody

The same antibody used for capture was labelled with peroxidase. It was diluted 1:5000 in 0.1 M PBS buffer containing 1% BSA.

1.3—Standard

The 20-114 peptide was used to prepare the series of calibration points. The peptide concentration was 1 mg/ml.

To plot the calibration curve, the standard had to be diluted in 0.1 M PBS/1% BSA according to the following table:

| Points | Concentration (ng/ml) |
|---|---|
| 1 | 125.00 |
| 2 | 62.50 |
| 3 | 31.25 |
| 4 | 15.62 |
| 5 | 7.81 |
| 6 | 3.90 |
| 7 | 1.95 |
| 8 | 0.97 |
| 9 | 0.48 |
| 10 | 0.24 |
| 11 | 0.12 |

1.4—Preparation of Samples

Fresh samples are recommended for the analysis. Plasma should be collected by established procedures and used in clinical laboratories. Where necessary, plasma may be stored between 2 and 8° C. for up to one week. Samples which are stored frozen may be used for a longer period.

Samples were diluted according to their source:

Plasma from normolipidic subjects: 1.3 to 4-fold.

Plasma from hypertriglyceridemic subjects: from 2 to 16-fold.

HDL prepared from plasma by ultracentrifugation: from 4 to 128-fold. Dilutions were in PBS/1% BSA.

2. Protocol

“Coating”: the unlabelled anti-AA4RP capture antibody was incubated overnight at room temperature at 100 μl/well (96-well microtiter plate).

Washing of plate: antibodies not bound on the plate were eliminated by washing the plate four times with 0.1 M PBS.

Loading of samples and calibration points: the peptide calibration solutions, human plasma and HDL were added at 100 μl per well, then incubated at 37° C. for 2 hours.

Wash: excess peptide and AA4RP from the biological samples which did not bind to the anti-AA4RP capture antibodies were eliminated by successively washing the microtiter plate four times.

Detection: detection was with peroxidase-labelled anti-AA4RP antibody diluted 1/5000 in PBS/1% BSA. 100 μl of preparation were added to each well of the microtiter plate. Incubation was at 37° C. for 2 hours.

Wash: excess anti-AA4RP detection antibody was eliminated by washing the plate four times with 0.1 M PBS.

Development of enzymo-colorimetric reaction: carried out by addition of 100 μl of o-phenylenediamine dihydrochloride (OPD) substrate per well, after which the reaction was developed for 30 minutes in the dark.

Stop reaction: by addition of 100 μl of 1 N HCl per well.

Read absorbance: on a spectrophotometer set at 492 nm.

3. Results and Interpretation

3.1—Calibration Curve

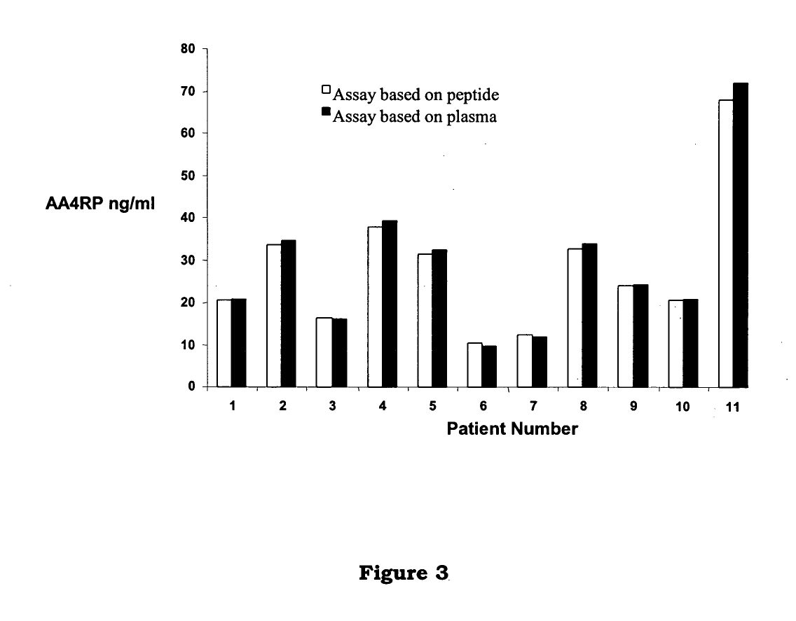

The calibration curve was made according to a function of four unknowns representing the increase in optical density as a function of the concentration of the different dilutions of the calibration points. It is a sigmoid curve characteristic of the ELISA immunoenzymatic assay (FIG. 2).

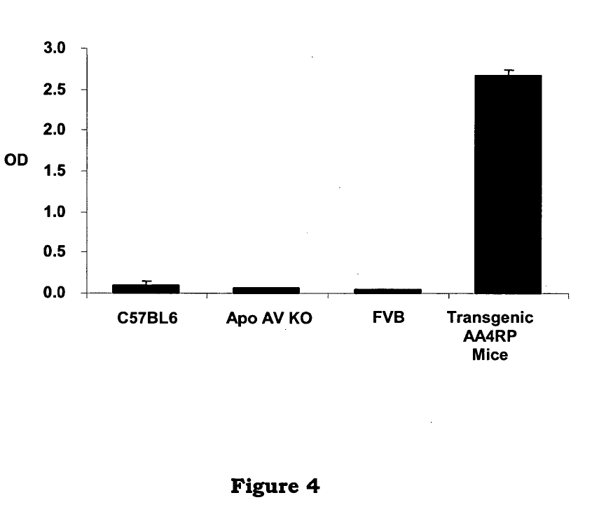

The curves representative of different dilutions of the 20-114 peptide, the normolipidic human plasma and HDL overlap, indicating that the anti-AA4RP antibody has the same affinity for the synthetic peptide and for native AA4RP in human plasma or lipoproteins. Furthermore, the assay of AA4RP in several subjects using a series of calibration points prepared either from the peptide or from pooled human plasma, gave significantly similar results for AA4RP concentration (FIG. 3).

3.2—Working Range and Limit of Detection

It is approximately 2 Log, between 1 and 62 ng/ml, which enables the assay of samples having very different AA4RP concentrations. The limit of detection is approximately 1 ng/ml, which indicates that the assay has very high sensitivity.

3.3—Normal Values

The assay of AA4RP in a population of normolipidic subjects with triglyceride levels not exceeding 1 mg/ml gave values of apolipoprotein concentration ranging from 9.84 to 29.44 ng/ml.

### Example 4

### Correlation Between AA4RP Concentration in Different Plasma Compartments and Total Triglyceride Levels

1. Protocol

The correlations between plasma triglyceride levels and concentrations of AA4RP in plasma or of AA4RP in non-HDL lipoproteins (VLDL, IDL and LDL) or of AA4RP in HDL lipoproteins were determined in a population with triglyceride levels comprised between 0.5 and 4 mg/ml.

1.1—Preparation of HDL and Non-HDL Lipoproteins

HDL and non-HDL lipoproteins were prepared by precipitation of the latter by addition of phosphotungstic acid and Mg2+ to plasma from different subjects (selective precipitation of apo B-containing lipoproteins, i.e. VLDL, IDL and LDL) (Burstein, Scholnick et al. 1970; Lopes-Virella, Stone et al. 1977). Briefly, the plasma was incubated for 10 minutes at room temperature in a phosphotungstate solution, then centrifuged at 2500 rpm for 15 minutes and finally the supernatant containing HDL was separated from the pellet containing non-HDL lipoproteins.

1.2—Assay of Triglycerides

Plasma triglycerides were assayed by the enzymo-colorimetric method, against a calibration curve prepared with CFAS lipid calibrator, Ref. 759350 (Boehringer Mannheim GmbH, Germany). The calibration curve covered a concentration range of 16 to 500 μg/ml. 100 μl of each sample dilution or calibration standard were deposited in each well of a 96-well titration plate. Next, 200 μl of triglyceride reagent Ref. 701912 (Boehringer Mannheim GmbH, Germany) were added to each well and the plate was incubated at 37° C. for 30 min. Optical densites (OD) were read on a spectrophotometer set at 492 nm. Triglyceride concentrations in each sample were calculated from a standard curve plotted as a linear equation y=ax+b, where y represents OD and x represents triglyceride concentration.

1.3—Assay of Total AA4RP, in HDL Lipoproteins and in Non-HDL Lipoproteins:

Total AA4RP levels in plasma and in HDL lipoproteins were assayed according to the protocol in example 3. The AA4RP concentration in non-HDL lipoproteins was calculated as the difference between the total plasma AAR4RP concentration and that of HDL lipoproteins.

2.—Results and Interpretation:

No correlation was observed between the AA4RP concentration in plasma and that of total triglycerides or triglycerides of non-HDL lipoproteins. On the other hand, a significant negative correlation was found between the concentration of the protein and that of triglycerides in HDL lipoproteins (r=0.5, p<0.001). This finding indicates that the greater the increase in triglyceride levels, the more the AA4RP concentration in HDL diminishes, thus confirming the anti-atherogenic role of HDL and consequently of AA4RP which is present in this type of lipoprotein.

### Example 5

### Quantification of AA4RP in Mice Transgenic for Human AA4RP

1.—Protocol:

The AA4RP concentration was determined in normal C57BL/6 mice, nontransgenic FVB mice, AA4RP knock-out mice (KO) with a FVB genetic background and mice transgenic for human AA4RP (FVB genetic background). The assay protocol was identical to that described in example 3.

2.—Results and Interpretation:

FIG. 4 shows that the assay detected AA4RP solely in the mice transgenic for human AA4RP, demonstrating the specificity of the method between man and mouse. This is very interesting, since it is possible to quantify exogenous AA4RP in modified animal models without contamination by the endogenous protein, thus allowing to distinguish between the effect of the animal's endogenous AA4RP and that of exogenous origin.

### REFERENCES

Axen, R., J. Porath et al. (1967). “Chemical coupling of peptides and proteins to polysaccharides by means of cyanogen halides.” Nature 214(95): 1302-4.

Bassiri, R. M., Dvorak J. and Utiger R. D. (1979). Thyrotropin-releasing hormone. Methods of hormone radioimmunoassay. B. M. In: Jaffe and Behrman H. R. (Eds). New York, N.Y. Academic Press: p: 46.

Burstein, M., H. R. Scholnick et al. (1970). “Rapid method for the isolation of lipoproteins from human serum by precipitation with polyanions.” J. Lipid Res. 11(6): 583-95.

Duverger, N., G. Tremp et al. (1996). “Protection against atherogenesis in mice mediated by human apolipoprotein A-IV.” Science 273(5277): 966-8.

Harlow, E. a. L. D., Ed. (1988). Antibodies: a Laboratory Manual. New York, Cold Spring Harbor Laboratory Publications.

Hokanson, J. E. and M. A. Austin (1996). “Plasma triglyceride level is a risk factor for cardiovascular disease independent of high-density lipoprotein cholesterol level: a meta-analysis of population-based prospective studies.” J. Cardiovasc. Risk 3(2): 213-9.

Huang, Y., X. Q. Liu et al. (1998). “Overexpression and accumulation of apolipoprotein E as a cause of hypertriglyceridemia.” J. Biol. Chem. 273(41): 26388-93.

Jones, P. T., Dear P. H., Foote J., Neuberger M. S. and Winter G. (1986). “Replacing the complementarity-determining regions in a human antibody with those from a mouse.” Nature 321: 522-525.

Kohler, G. and C. Milstein (1975). “Continuous cultures of fused cells secreting antibody of predefined specificity.” Nature 256(5517): 495-7.

Lopes-Virella, M. F. P. Stone et al. (1977). “Cholesterol determination in high-density lipoproteins separated by three different methods.” Clin. Chem. 23(5): 882-4.

Lowry O. H., R. J., Farr A. L. and Randall R. J. (1951). “Protein measurement with folin phenol reagent.” J. Biol. Chem. 193: 265-275.

Lusis, A. J. (2000). “Atherosclerosis.” Nature 407(6801): 233-41.

Merrifield, R. B. (1963). “Solid phase peptide synthesis. The synthesis of a tetrapeptide.” J. Am. Chem. Soc 85: 2149-2154.

Pennacchio, L. A., M. Olivier et al. (2001). “An apolipoprotein influencing triglycerides in humans and mice revealed by comparative sequencing.” Science 294(5540): 169-73.

Plump, A. S., J. D. Smith et al. (1992). “Severe hypercholesterolemia and atherosclerosis in apolipoprotein E-deficient mice created by homologous recombination in ES cells.” Cell 71(2): 343-53.

Riechmann, L., Clark M., Waldmann H. and Winter G. (1988). “Monoclonal antibody therapeutic trials in seven patients with T-cell lymphoma.” Nature 332: 323-327.

Rubin, E. M., R. M. Krauss et al. (1991). “Inhibition of early atherogenesis in transgenic mice by human apolipoprotein AI.” Nature 353(6341): 265-7.

Rubin, E. M. and A. Tall (2000). “Perspectives for vascular genomics.” Nature 407(6801): 265-9.

Towbin, H., T. Staehelin et al. (1979). “Electrophoretic transfer of proteins from polyacrylamide gels to nitrocellulose sheets: procedure and some applications.” Proc. Natl. Acad. Sci. USA 76(9): 4350-4.

Vaitukaitis, J., J. B. Robbins et al. (1971). “A method for producing specific antisera with small doses of immunogen.” J. Clin. Endocrinol. Metab. 33(6): 988-91.

Ward, E. S., D. Gussow et al. (1989). “Binding activities of a repertoire of single immunoglobulin variable domains secreted from Escherichia coli.” Nature 341(6242): 544-6.

1-19. canceled.

20. Substantially pure synthetic peptide, wherein it consists in the amino acid sequence SEQ ID NO: 2 or an immunogenic fragment or a derivative thereof.

21. Peptide according to claim 20, wherein it consists in 5 to 95 aminoacids, preferably from 10 to 95 aminoacids.

22. Peptide according to claim 20, wherein the peptide is soluble or complexed with a carrier molecule such as KLH, serum albumin or a bead.

23. Antibody specific of a peptide consisting in the amino acid sequence SEQ ID NO: 2 or an immunogenic fragment or a derivative thereof, or a fragment or derivative of said antibody having substantially the same antigenic specificity.

24. Antibody according to claim 23, wherein it is produced by immunizing a non-human animal with a peptide consisting in the amino acid sequence SEQ ID NO: 2 or an immunogenic fragment or a derivative thereof, and recovering antibodies or antibody-producing cells.

25. Antibody according to claim 23, wherein it is a polyclonal antibody.

26. Antibody according to claim 23, wherein it is a monoclonal antibody.

27. Method for producing a specific anti-AA4RP antibody, comprising administering to a non-human animal a peptide consisting in the amino acid sequence SEQ ID NO: 2 or an immunogenic fragment or a derivative thereof and recovering antibodies or antibody-producing cells.

28. Method for detecting AA4RP in a biological sample comprising contacting the sample with an antibody according to claim 23, and detecting the presence of antigen-antibody immune complexes.

29. Method for detecting AA4RP in a biological sample, wherein it comprises contacting the sample with an antibody according to claim 23, and the presence of antigen-antibody immune complexes is determined by EIA, RIA, nephelometry, turbidimetry, quantitative immunoblot, calorimetry, interferometry, surface resonance or force field measurement.

30. Method for detecting AA4RP in a biological sample, wherein it comprises contacting the sample with an antibody according to claim 23, and the presence of antigen-antibody immune complexes is determined by an ELISA test.

31. Method for quantifying AA4RP in a biological sample, comprising contacting said sample with an antibody according to claim 23, and quantifying the AAR4P-antibody immune complexes.

32. Method for quantifying AA4RP in a biological sample, comprising contacting said sample with an antibody according to claim 23, and quantifying the AAR4P-antibody immune complexes by an ELISA method.

33. Method for detecting AA4RP in a biological sample comprising contacting the sample with an antibody according to claim 23, and detecting the presence of antigen-antibody immune complexes, wherein the sample is a sample of blood, serum, interstitial fluid or a tissue or cellular extract.

34. Method for detecting the presence of a predisposition or a risk of developing a disorder of lipid metabolism in a subject, comprising quantifying in vitro or ex vivo the levels of AA4RP in a sample from a subject, with an antibody according to claim 23.

35. Method for monitoring a treatment for correcting disorders of lipid metabolism in a subject, comprising measuring the quantities of AA4RP in vitro or ex vivo in a sample from said subject, by means of an antibody according to claim 23.

36. Antibody according to claim 23, wherein the antibody is coupled to a heterologous fragment such as a toxin, a marker, a medicament or any other therapeutic agent.

37. Pharmaceutical composition comprising an antibody according to claim 23 and a pharmaceutically acceptable excipient or vehicle.

38. Kit comprising a peptide according to claim 20.

39. Kit comprising an antibody according to claim 23.
